(19) United States
(12) Patent Application Publication
(10) Pub. No.: US 2026/0185028 A1
(43) Pub. Date: Jul. 2, 2026

(54) INSERT FOR CULTURING TISSUE AND METHOD OF CULTURING TISSUE USING SAID INSERT

(71) Applicant: NOTTINGHAM TRENT UNIVERSITY, Nottinham (GB)

(72) Inventors: Lívia Santos, Nottingham (GB); Yang Wei, Nottingham (GB)

(21) Appl. No.: 19/131,645

(22) PCT Filed: Nov. 21, 2023
(86) PCT No.: PCT/GB2023/053038

§ 371 Date: May 21, 2025

(30) Foreign Application Priority Data
Nov. 21, 2022 (GB) 2217400.7
Oct. 4, 2023 (GB) 2315239.0

(51) Int. Cl. C12M 3/00; C12M 1/00; C12M 1/34

(52) U.S. Cl. CPC C12M 23/44; C12M 21/08; C12M 23/34; C12M 23/48; C12M 41/40

(57) ABSTRACT

There is provided an insert (100) for culturing tissue comprising: abase (110) comprising a groove (140) configured to provide a guide for adding cells to be cultured; a first pillar (120) upstanding from the base and configured to support a first anchor; and a second pillar (120) upstanding from the base (110) and configured to support a second anchor. The insert (100) is configured so that cells may be cultured to form tissue between the first and second pillars (120) using the anchors for support.

The invention relates to a cell culture insert, in particular to a cell culture insert for use in a method of 3D cell culture.

### BACKGROUND TO THE INVENTION

Culturing cells and tissue in the lab has many potential applications in drug discovery, medicine and in other areas. When conducting research into drug discovery, one of the most common mechanisms is to use an animal model. However, this has many drawbacks. Animals are poor predictors of human response to drugs. Due to the genetic and metabolic differences between lab animals such as mice and humans there is a very low success rate, with less than 5% of treatments tested in animals successfully making it into human trials.

An alternative to animal models is to use engineered human tissue. However, current methods of 3D cell culture involve very expensive and complex bioreactors that are not accessible for many labs either due to expense or the ability to run and maintain them. Also, current methods of 3D cell culture are low throughput since they typically generate 2 or 3 tissue engineered muscle at a time. This does not meet the needs of pharma since they require high-throughput platforms to enable screening of a vast number of candidate drugs. Current high-throughput methods of muscle culture involve culturing cells in a 2D orientation (monolayer). Culturing muscle cells in 2D means that the structure of the tissue is not the same as would be found in vivo. Thus, using a 2D culture means that the model of human disease is not as accurate, or the engineered tissue will not accurately match the tissue it is being transplanted into when used to treat a subject.

Outside of research, implanting engineered tissues may be used in the treatment of conditions like volumetric muscle loss, oesophageal defects, abdominal defects, or sports injuries. However, current 2D methods of tissue culturing are not well suited for this. For example, with muscle tissues 2D culture does not exactly replicate the structure of a subject's muscle or exhibit functional maturation markers like contractility. Additionally, current engineered muscle tissue culturing techniques are limited in the size of the tissue that can be grown, with a maximum length of around 2 cm. Engineered muscle tissue culturing techniques are also expensive, and require high end specialist equipment, which is inhibitive for many institutions.

Other uses for cell culture include the production of lab based meat products. This is a field experiencing a lot of growth at the moment, as lab based meat products have several advantages over using animals. For example, lab based meat products do not involve killing livestock and so can be considered to be more ethical, additionally, the equipment used to produce the meat can be far more space efficient giving lab based meat potential application in environments where space is at a premium. Current methods of lab based meat production are currently extremely expensive, require large amounts of space and face other challenges in becoming a viable food source.

The present invention aims to address or ameliorate one or more of these issues.

### SUMMARY OF THE INVENTION

According to a first aspect of the present the invention there is provided an insert for culturing tissue comprising:
a base comprising a groove configured to provide a guide for adding cells to be cultured;
a first pillar upstanding from the base and configured to support a first anchor; and
a second pillar upstanding from the base and configured to support a second anchor,
wherein the insert is configured so that cells may be cultured to form tissue between the first and second pillars using the anchors for support.

The base comprises a groove configured to provide a guide for adding cells to be cultured. The cells may be added into the groove itself, or be seeded on top of a hydrogel previously casted in the groove. The groove may extend between the first and second pillars and pass through the centre of the base. As mentioned, the groove provides a guide for the user when adding the cells to the insert. Advantageously, this enables the user to seed the insert with the cells such that during culture they grow and form tissue between the 2 anchors. The insert used for the initial seeding and growth of the cell culture may be referred to as a primary insert.

Advantageously, tissue cultured using the insert has a structure extremely similar to that of the equivalent tissue in an organism. This tissue can then be used as a model for research or implanted into a subject to treat a disease or injury. Further advantageously, the insert may be scalable and reliable to produce, reducing the inhibitive cost of research.

The position of the first and/or second anchor may be configured to be adjusted to stretch the tissue. Advantageously, this may allow for tissue growth using the insert to more closely mimic tissue growth in vivo.

The location of pillars on the base may vary and the pillars can be located closer together or further away from each other as desired, during manufacture of the insert. Additionally or alternatively, at least one pillar may be adjustable. The adjustable pillar may be moved closer or further away from the other pillar. Alternatively, the angle of the adjustable pillar may be changed.

The groove may further comprise a recess. Advantageously, the recess enables the user to coculture additional cell types.

The groove may be located such that it extends between the points where the first and second pillars meet the base. The groove may be sized such that it can receive cells suspended in a hydrogel. If there are more than two pillars present, the groove may extend between the points where each of the pillars meet the base. In one embodiment there may be up to 12 pillars attached to the base. In this instance, the groove may extend from the centre out to each pillar.

In one embodiment, the cells may be embedded in the hydrogel.

In one embodiment, the tissue is muscle tissue. In one embodiment, the muscle may be human muscle tissue. In one embodiment, the tissue may be livestock tissue and wherein the livestock may be a cow, pig, sheep, or chicken.

In one embodiment, the pillars are made of a biocompatible material, optionally wherein the biocompatible material is a biocompatible resin. The biocompatible material is advantageous as it does not impact cell culture other than providing the support for the tissue to grow. Examples of materials that can be used for the base and the pillars include, but are not limited to organic materials, e.g., resin, polycaprolactone, silicone, and inorganic materials e.g., silicon, silica, glass, metals, calcium phosphate, and the mixture of organic and inorganic materials.

In one embodiment, the pillars comprise a biodegradable material. Biodegradable materials are advantageous as they will break down over time into non-toxic components that will not interfere with cell growth.

In one embodiment, the anchors comprise a biocompatible non-woven textile. Similarly, the biocompatible non-woven textile is advantageous as it supports the tissue during culture and allows the tissue to be stretched in a simulation of in vivo tissue.

In one embodiment, the anchors comprise a biodegradable non-woven textile. A biodegradable anchor may be advantageous as it can be used to fix an implant of the tissue into a subject without interfering with normal physiological function, and then breakdown into non-toxic components. Examples of materials that can be used for the anchors include but are not limited to non-woven or fabric woven. These fabrics could be synthesised or made out from naturals materials, e.g., cellulose and silk. These fabrics could have a coating or could be used on its own without coating. These fabrics could be stretchable or non-stretchable.

The anchors may have one or more holes pierced in them. These holes may provide the means of attachment to the pillars as the pillars pierce the hole to secure the anchors in place. Different holes may be used as the attachment point at different points during cell culture.

In one embodiment, the anchors are removeable from the pillars. In one embodiment, the anchors may be removable during cell culture to facilitate transfer of the cultured tissue to a second set of pillars. The second set of pillars may be on the same insert, or a different insert.

Each of the anchors may comprise one or more holes. These holes may be used to attach the anchors to the respective pillars.

The position of the anchors may be adjusted by using the one or more holes in the anchor to stretch the engineered tissue. Alternatively, engineered tissue may be stretched by transferring it to an insert with pillars located further apart or by sliding one the pillars further apart. The position of the anchors can be adjusted manually, automatically and semi-automatically.

In one embodiment, the insert may be stackable with other units of the insert. The base of the insert may comprise one or more recesses. These one or more recesses may be located on the underside of the base. Each recess may be configured to receive a pillar from another insert. The recesses may be disposed along a plane perpendicular to the groove, such that a plurality of inserts are stacked with 90 degree rotation between each layer (i.e. in an ABABAB . . . formation). Alternatively, the recesses may be disposed on the underside of the base such that they correspond with the upstanding pillars of the same insert. This allows for the inserts to be stacked without rotation between each layer (i.e. in an AAAAA formation).

Other mechanisms for storing and/or stacking the inserts may be provided. For example, each of the plurality of inserts may act as a removable ‘shelf’ within a larger structure configured to receive a plurality of inserts. Advantageously, this allows for individual inserts to be removed without disturbing other nearby inserts.

The insert may further comprise one or more spacers. Each of these one or more spacers may be disposed at the bottom of a pillar. In an embodiment where two pairs of pillars are provided, one pair of pillars may be provided with the spacers. Advantageously, providing a spacer at the base of a pillar may act to raise the supported anchor, and so provide the tissue being grown to be grown in suspension. Advantageously, the cell culture being grown in suspension facilitates the exchange of nutrients and gases during the growing process.

The spacer may comprise a bead at the bottom of the pillar. The spacer may comprise a removable collar configured to be placed over a pillar. Advantageously, this may allow for the insert to be customisable by an end user selecting which pillars raise the height of the associated anchor in use.

The insert may further comprise a third pillar upstanding from the base and configured to support an anchor; and a fourth pillar upstanding from the base and configured to support an anchor. The third and fourth pillar may be disposed along the plane perpendicular to the groove. Optionally, at least one of the third and fourth pillar may comprise a spacer at the base of the pillar in question, such that the supported anchor is raised above the base of the insert. The third and fourth pillars may be referred to as a second pair of pillars, such that the insert comprises two pairs of pillars with a rotational symmetry of 4. The insert as a whole may have a rotational symmetry of 2, accounting for the groove, but the pillars may have a rotational symmetry of 4.

Advantageously, providing a third and fourth pillar in the arrangement described allows for a user to change the pillars supporting the anchors to change the direction of the tension applied to the tissue being grown using the insert. The optional addition of a spacer provides a difference between the first and second pillars and the third and fourth pillars. By supporting the anchors with the third and fourth pillars, the tissue is then suspended in the hydrogel, rather than sitting within the groove of the insert.

Additionally or alternatively, the insert may comprise one or more additional pillars configured to support one or more corresponding additional anchors, optionally or preferably wherein the first, second and one or more additional pillars are spaced equidistant around the base of the insert. In contrast to the embodiment described with the ‘two pair’ system, each of the pillars may support a distinct anchor rather than a user transferring an anchor from one.

In one embodiment, the first and second pillars may be arranged at least 10 mm, 20 mm apart, at least 22 mm apart, at least 25 mm apart, at least 28 mm apart, at least 30 mm apart, at least 32 mm apart, at least 35 mm apart, at least 38 mm apart, at least 40 mm apart.

Surprisingly, using the insert to culture cells allows for longer pieces of tissue to be grown than was previously possible. Longer pieces of tissue are advantageous for use in medicine because they are more versatile and can be used to treat a wider range of conditions.

In one embodiment, the pillars may be disposed at different heights and angles in the same or different planes. Advantageously, this may alter the positions of the anchors and promote directional growth.

In one embodiment, the insert comprises a force transducer, optionally or preferably wherein the force transducer is attached to either the first or second anchor. The force transducer may be used to measure contractility and in so doing demonstrate that the muscle is functioning correctly. The force transducer may be manufactured at the same time as the insert or made separately and then attached subsequently. The contractility could be measured using a physical sensor e.g., a piezoresistive sensor or an optic fibre sensor e.g., an optic fibre. The transducer interface circuit may be integrated as part of the insert or be located outside the cell culture well-plates.

In one embodiment, the insert is configured to provide electrical stimulation to the tissue grown using the inset. The electrical stimulation may be provided by a lid for the insert. The lid may comprise a pair of electrodes disposed along the surface, configured to contact the tissue being grown in the insert. The pair of electrodes may be connected to an external power source.

According to a second aspect of the present invention, there is provided a system for culturing tissue, comprising:
the insert according to any embodiment of the first aspect; and
a plate comprising at least one well;
wherein the at least one well contains the insert and the engineered tissue is suspended in the insert.

The system may further comprise a lid configured to cover at least the well storing the insert. The lid may comprise two or more electrodes configured to provide electrical stimulation to the contents of the well. The electrode may comprise metallic materials, such as silver and copper, or conductive polymers, such as PEDOT:PSS, or non-metallic materials, such as carbon, graphene, or graphite. The electrode may be rigid, semi-flexible, or flexible. The electrodes may be provided in pairs. Alternatively, an odd number of electrodes may be provided. In the odd number configuration, a common ground electrode may be shared with other electrodes, whereas in an even number configuration, each pair may have its own ground electrode. Each electrode pair may provide the same consistent stimulation. Alternatively, each pair may provide different levels stimulation at different intervals. The stimulation from each pair may be provided simultaneously or sequentially.

The system described herein may further comprise a secondary insert wherein the secondary insert comprises: a body comprising an aperture; a first pair of pillars upstanding from the body and configured to support a first pair of anchors; and a second pair of pillars upstanding from the body and configured to support a second pair of anchors; wherein the secondary insert is configured to receive one or more pairs of anchors removed from the pillars of the primary insert during growth of the tissue.

In one embodiment, the pairs of pillars are parallel. Advantageously, parallel pairs of pillars may be more space efficient than other alternative arrangements.

The secondary insert may comprise one pair of pillars, 2 pairs of pillars, 3 pairs of pillars, 4 pairs of pillars, 5 pairs of pillars, or more than 5 pairs of pillars.

The cells received on the secondary insert may be from more than one primary insert, or from the same primary insert.

The aperture of the secondary insert may be a groove. The aperture of the secondary insert may comprise multiple grooves.

In one embodiment, the cells may be cultured on the primary insert for at least 48 hours, 60 hours, 72 hours, 4 days, 5 days, 6 days, 1 week, 2 weeks, 3 weeks or 1 month before being transferred to the secondary insert.

In another aspect of the invention, there is provided an in vitro method of cell culture using the insert as described herein.

In one embodiment, the tissue is muscle tissue; optionally wherein the tissue is mammalian tissue; optionally wherein the tissue is human tissue; preferably wherein the tissue is human muscle tissue. In one embodiment, the tissue may be bird tissue; preferably wherein the tissue is from poultry.

Cells used in the method may be myocytes including cardiomyocytes, or other cells such as smooth muscle cells, myoblasts, myosatellite cells, fibroblasts, muscle progenitor cells, induced pluripotent stem cells (iPSCs), and tenocytes.

Additional cell types including adipocytes and/or motor neurone cells may also be cultured on the insert.

The cells may be cultured in a hydrogel. The hydrogel may comprise laminin, nidogen, collagen, fibrin, matrigel and heparan sulfate proteoglycans.

Cells may be prepared for culture with the insert by expanding and growing cells using conventional methods before transferring the cells into a hydrogel and seeding them onto the cell insert.

Cells may be seeded into the groove of the cell culture insert. Alternatively, cells may be seeded in co-culture, into the groove and onto the base of the insert, or on top of the hydrogel itself. Once the hydrogel has set, cell culture media may be added. The groove may comprise a recess. The recess may be configured for the co-culture of adipocytes and/or motor neurone cells.

During culture, the cells will integrate with the anchors of the insert. The cells will form a tissue with muscle fibres extending between the anchors.

In one embodiment, the insert comprises up to 12 anchors and the muscle tissue may form a patch.

In one embodiment, the cells may be transferred from the first and second pillars to a second pair of pillars comprising third and fourth pillars. This second pair of pillars may be upstanding from the base along a plane perpendicular to the plane defined by the first pair of pillars, as discussed with respect to the first aspect. Advantageously, transferring the cells to the second pair of pillars allows for uniaxial stretching and mechanical tension which acts as a maturation cue.

The cells may be cultured for at least 48 hours, 60 hours, 72 hours, 4 days, 5 days, 6 days, 1 week, 2 weeks, 3 weeks or 1 month.

In a further aspect of the invention, tissue cultured according to the methods described herein are used medicine.

In a further aspect of the invention, there is provided a method of treatment comprising implanting tissue cultured using the insert as described herein. The method may include implanting the anchor of the insert with the tissue into a subject in need thereof.

In another aspect of the invention, there is provided tissue cultured as described herein for use in the manufacture of a medicament.

In one embodiment, the tissue for use in medicine, or as in a method of treatment, or in the manufacture of a medicament has a length of at least 20 mm, at least 22 mm, at least 25 mm, at least 28 mm, at least 30 mm, at least 32 mm, at least 35 mm, at least 38 mm, at least 40 mm.

In one embodiment, tissue cultured for use in medicine is used for treating muscle disorders and injuries resulting from trauma. The muscle disorder may be abdominal wall or oesophageal defects.

Tissue cultured using the methods described herein can be used in medicine. The tissue can be implanted into a subject in need thereof to treat disease or injury. The medicine in question may be regenerative medicine.

The tissue can be implanted with the anchors. The anchors may be used to secure the tissue in position.

The tissue may be used to treat muscle disorders or conditions including: abdominal wall defects and oesophageal defects, or volumetric muscle loss derived from trauma. Other uses may include the treatment of: sarcopenia, Duchenne muscular dystrophy, Becker muscular dystrophy, DMD-associated dilated cardiomyopathy, Limb girdle muscular dystrophies (LGMD), LAMA2-related (merosin deficient) congenital muscular dystrophy (Emery-Dreifuss muscular dystrophy), Collagen VI-related muscular dystrophy (Bethlem myopathy, Ullrich congenital muscular dystrophy), a-Dystroglycanopathies (Walker-Warburg syndrome, muscle-eye-brain disease), Laminopathies, Distal muscular dystrophy, Myofibrillar myopathies, Nemaline myopathy, Central core myopathy, Centronuclear myopathy, Congenital fiber type disproportion, Multi/minicore myopathy, Cylindrical spirals myopathy, Mitochondrial myopathies, Glycogen storage diseases, Metabolic diseases, Inflammatory myopathies, Motor neurone diseases (for example Amyotrophic lateral sclerosis and Spinal muscular atrophy), Spinal and bulbar muscular atrophy, Charcot-Marie-Tooth disease, and Kennedy's disease.

In one aspect of the invention, the cell insert is used to culture meat products. The cells used in the production of meat products may be livestock cells. The cells may be bovine, ovine, poultry or swine cells.

The cell culture insert may be manufactured using additive manufacturing technology, e.g., 3D printing, or subtractive technology, e.g. milling. Alternatively, the insert may be made using moulding, forming, casting, machining and joining.

One or more inserts may be used in tandem. For example, a plurality of inserts may be placed within a single system, to produce multiple samples at the same time. Additionally, and/or alternatively, an insert may have more than one pair of pillars and corresponding anchors. Advantageously, this allows for production of the muscle tissue to be scaled up, for use in research, medicine, or cultured meat products.

Anchor: As used herein, the term anchor refers material that provides support for cells during culturing. The anchor may also be used to move the tissue once cultured for use in downstream experiments and to culture the tissue in suspension. The anchor may be made of a non-woven textile. The material forming the anchor may be a mesh.

Base: As used herein, the term base refers to the structure of the cell culture insert that provides support for the pillars. Cells may also be seeded onto the base. The base may be made of a biocompatible resin.

Cells: As used herein, the term cells refers to those cells that are cultured on the cell culture insert. The cells may be mammalian cells, preferably human cells. The cells may be from livestock. The cells may be from a cow, pig, sheep or chicken. The cells may be muscle cells. In one embodiment, the cells may be derived from established cell lines such as C2C12 or human myoblasts. In one embodiment, the cells may be derived from a patient. In one embodiment, the cells are stem cells, optionally induced pluripotent stem cells. The cells may be skeletal muscle cells, cardiomyocytes, smooth muscle cells or tenocytes.

Biodegradable: as used herein, biodegradable refers to a material that over time breaks down into non-toxic components.

Biocompatible material: as used herein, biocompatible material refers to a material that can be used to culture cells or implanted into a subject without causing any damage or otherwise interfering with cell or tissue function. The material may be a resin. The material may be a non-woven mesh.

Muscle disorders: the muscle disorders described herein may affect a mammal, in particular a human. The muscle disorders may include abdominal wall defects etc.

Embodiments of the invention will now be described in more detail, by way of example only, with reference to the accompanying drawings, in which:

FIG. 1 illustrates a cell culture insert according to an embodiment of the present invention;

FIG. 2 illustrates a cell culture insert according to an alternative embodiment of the present invention;

FIG. 3a illustrates a plurality of cell culture inserts according to an embodiment of the present invention;

FIG. 3b illustrates the cell culture inserts with muscle growth in situ;

FIG. 4 illustrates a cell culture insert having a force transducer set up;

FIGS. 5a and 5b illustrate evidence of the muscle growth engineered with the cell culture inserts of embodiments of the present invention.

FIGS. 6a to 6c illustrate three possible configurations of the pillars on the inserts.

### EXAMPLE METHODS AND DETAILED DESCRIPTION OF THE FIGURES

FIGS. 1 and 2 illustrates the cell culture insert 100. The insert comprises a base 110. The base 110 comprises a biocompatible resin and provides a support structure for the other components of the insert 100. There are two pillars 120 upstanding from the base 110. In the illustrated example, the pillars 120 are formed contiguously with the base and provide attachment points for first and second anchors 130 (FIG. 2). The anchors 130 are formed of a biocompatible non-woven textile and provide a scaffold for the cells to use when forming tissue. The insert 100 also comprises a groove 140. The groove 140 is formed into the base 110 and extends between the first and second pillars 120. The groove is configured to provide the user with a guide for applying the cells to the base 110. During use, the cells are added to the base 110 between the first and second pillars 120 into the groove 140.

The insert 100 of FIG. 1 is attached to a stand 150. This stand is used during the manufacturing process of the insert (e.g., as part of the 3D model print, or the mould), but is removed before use with culturing cells.

FIGS. 3a and 3b illustrates the cell culture inserts 100 during the muscle growth process. Like the inserts of FIGS. 1 and 2, each insert 100 comprises a base 110, pair of pillars 120, anchors 130 and a groove 140. Each insert 100 contains cells suspended in hydrogel 320, on a 6 well plate 310. An example composition of the hydrogel 320 is discussed below. FIG. 3b illustrates one of the inserts of FIG. 3a, further along the muscle growth process. Muscle tissue 430 is present between the two pillars 120, using the hydrogel 320 as a scaffold for growth.

FIG. 4 illustrates the muscle tissue 430 being tested for contractability using a force transducer 500. The insert 100 comprises the same features as FIGS. 1 to 3. In the illustrated embodiment, the force transducer 500 is an ‘off the shelf’ component. It is envisaged that a version of the insert 100 may include elements of a force transducer integrated into one or more of the components (e.g., the pillars 120).

FIG. 5 is provided as confirmation of the growth of the muscle tissue 430 growth. FIG. 5 illustrates the muscle structure under a microscope, with two different stains 550, 560. In 550, the nuclei of aligned myotubes was stained with DAPI. In 560, the cytoskeleton of aligned myotubes was stained with phalloidin.

FIG. 6a illustrates a schematic of the insert 100 of FIGS. 1 and 2. In the insert 100 as depicted in FIG. 6A, it can be seen that the pillars 120 are upstanding from the groove 140 set within the base 110. FIG. 6 does not illustrate the anchors 130 illustrated in FIG. 2.

FIG. 6b illustrates an alternative insert 600. Like the insert 100 of FIG. 6A, insert 600 comprises a base 110, with pillars 120 upstanding from the base 120, in particular upstanding from within the groove 140. In contrast with the insert 100 of FIG. 6A, insert 600 comprises a recess 610 within the groove 140. This recess 610 is configured to facilitate the co culture of a variety of cell types, which adipocytes and motor neurone. In this illustrated example, the recess 610 is formed in the centre of the base 110, and comprises two semicircle indents into the base 110 extending from the groove 140.

FIG. 6c illustrates an alternative insert 650. Like the insert 100 of FIG. 6A, insert 650 comprises a base 110, with pillars 120 upstanding from the base 120, in particular upstanding from within the groove 640. In contrast with the insert 650 of FIG. 6A, a plurality of pillars 120 are provided, and as such the configuration of the groove 640 differs from that of groove 140 (FIG. 6A). In the illustrated example of FIG. 6C, twelve pillars 120 are provided, and are spaced equidistant around the rim of the insert 650. As such, the groove 640 resembles a twelve pointed star shape; and can be visualised as each pair of pillars 120 having a portion of the groove 740 extending between them. Anchors 140 that would be associated with each pillar 120 are not presented for the sake of clarity. An advantage of this more complex configuration is that tissue can be grown as a “patch”, which may be useful for different tissue types such as cardiac tissue.

FIG. 7 illustrates an alternative insert 700. Like the insert 100 of FIG. 6A, insert 600 comprises a base 110, with pillars 120 upstanding from the base 120, in particular upstanding from within the groove 140. In contrast with insert 100 of FIG. 6A, insert 700 comprises a second pair of pillars 125. These are disposed in the plane perpendicular to the groove 140, such that the groove 140 bisects the path between the second pair of pillars 125. The second pair of pillars 125 each comprise a spacer 170 disposed at the base of each pillar 125. The spacer 170 at the base of each pillar 125 acts to raise the supported anchor (not pictured for clarity). In the illustrated example, supporting the anchors with the second pair of pillars 125 would lead to the tissue being suspended in the hydrogel, rather than sitting within the groove 140 of the insert 700. A user may transfer the anchors from the first pair of pillars 120 to the second pair of pillars 125 to enable this change of growth conditions during the culturing of the tissue. Insert 700 further comprises a pair of apertures 150 in the base 110 of the insert 700. These apertures 150 allow for microscopic observation of the tissues being cultured, particularly when in suspension using the second pair of pillars 125. In the illustrated example the apertures 150 are circular, but they may be any shape. Similarly, a single aperture 150 may be provided. A plurality of viewing apertures 150 may be provided. The insert 700 also comprises aperture 160. Aperture 160 is configured to allow the release of gas bubbles that may become trapped underneath the insert 700. These gas bubbles would act to prevent the insert 700 from floating. Provision of aperture 160 reduces the risk of this occurring.

FIG. 8 illustrates an embodiment of a secondary insert 800. Similar to the insert 700 of FIG. 7, which may be referred to as a primary insert 700, the secondary insert 800 comprises a pair of pillars 120 upstanding from a base 110. In the illustrated embodiment, the secondary insert 800 comprises a plurality of pairs of pillars 120. In contrast with the primary insert 700, the secondary insert 800 comprises a central aperture 180 in the base 110 wherein the central aperture 180 is configured to receive the cultured tissue, rather than a groove 140 for receipt of cells to be cultured. Pillars 120 of the secondary insert 800 are configured to receive a pair of anchors during the growth of the tissue. The plurality of pillars 120 allow for a plurality of primary inserts 700 to be associated with each secondary insert 800. This improves the scalability of the tissue culturing. In the illustrated embodiment, a 3:1 scaling could be achieved.

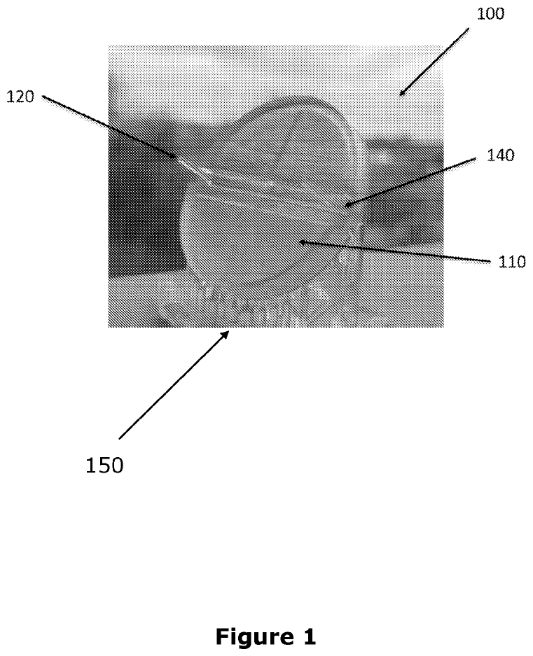

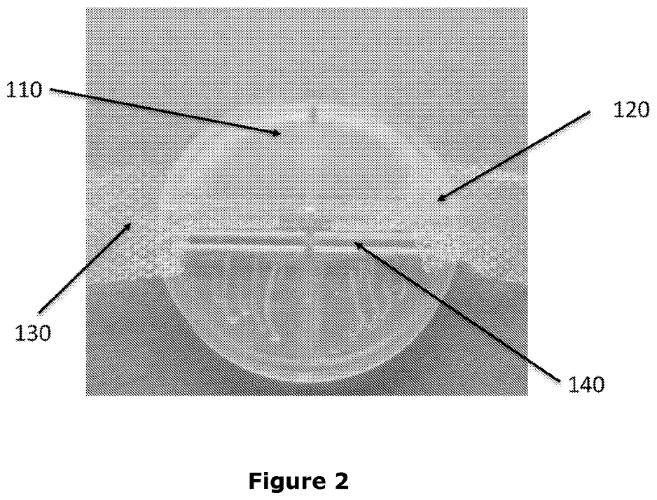

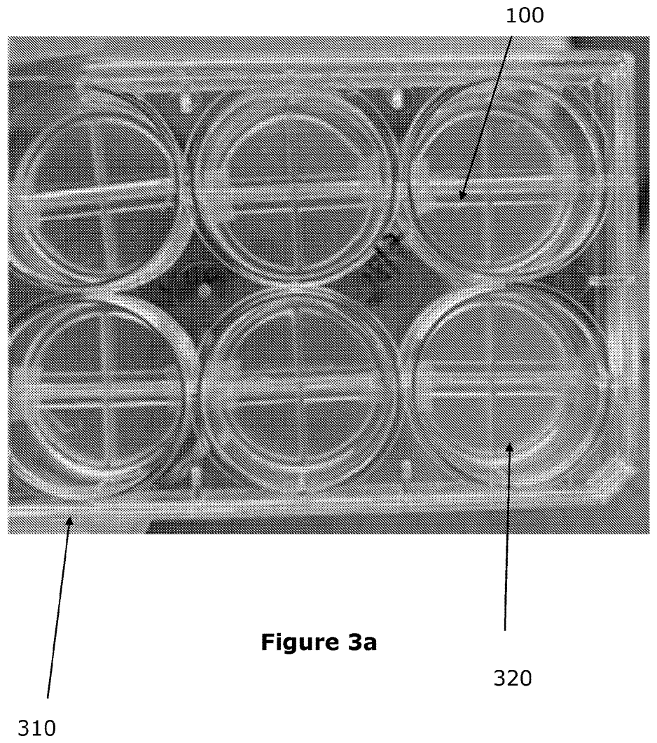

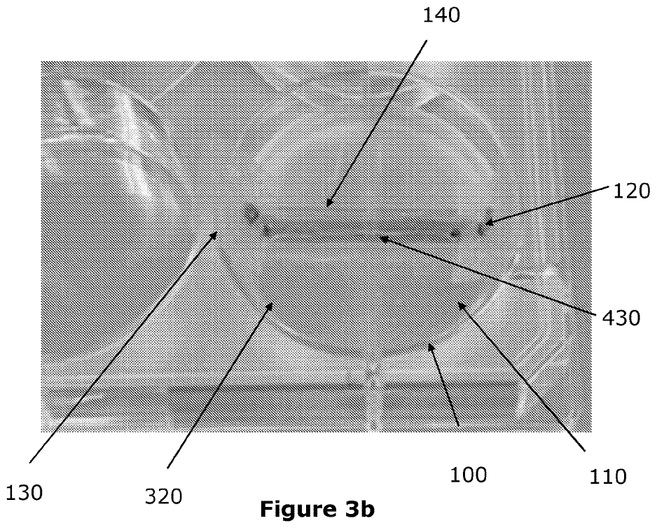

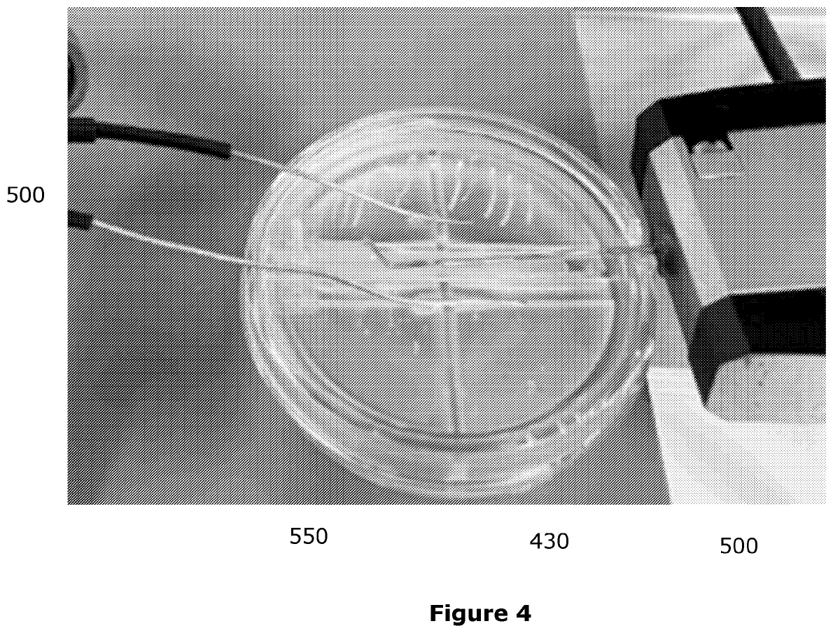

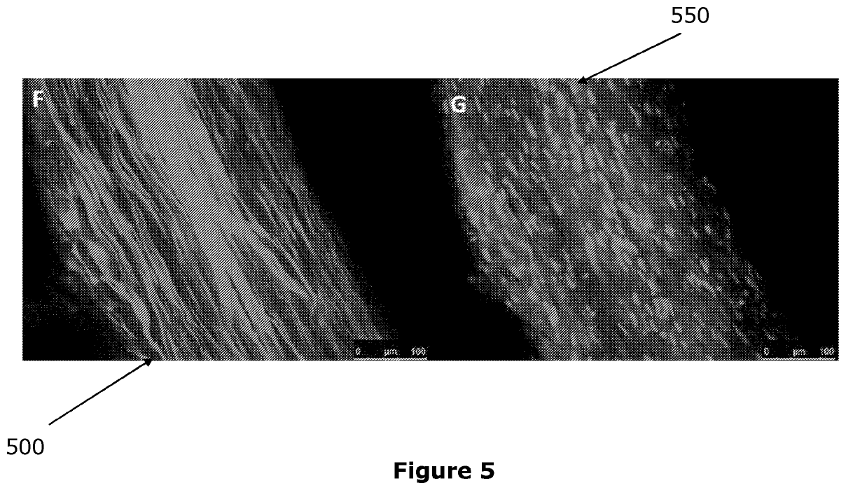

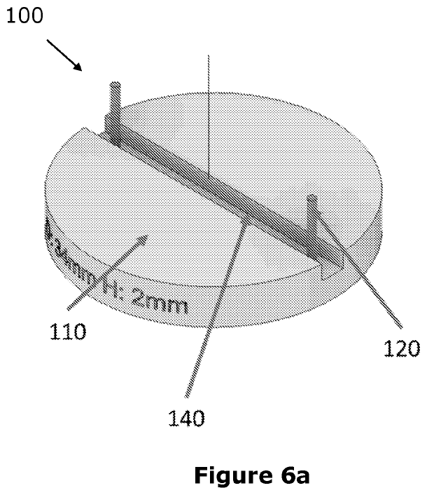

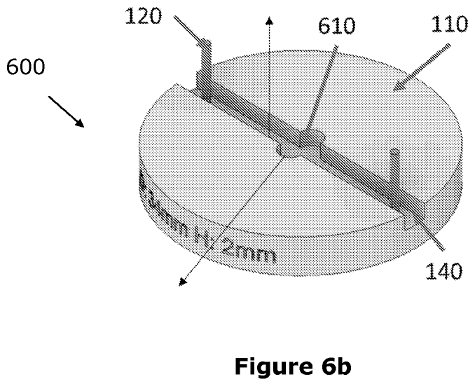

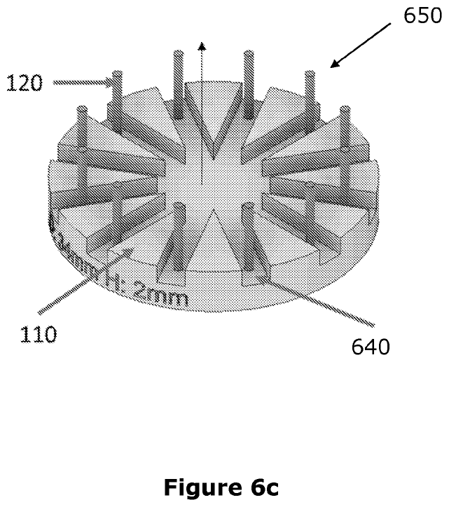

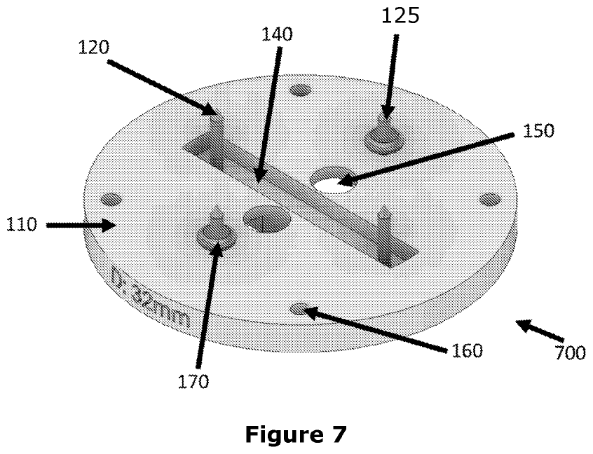

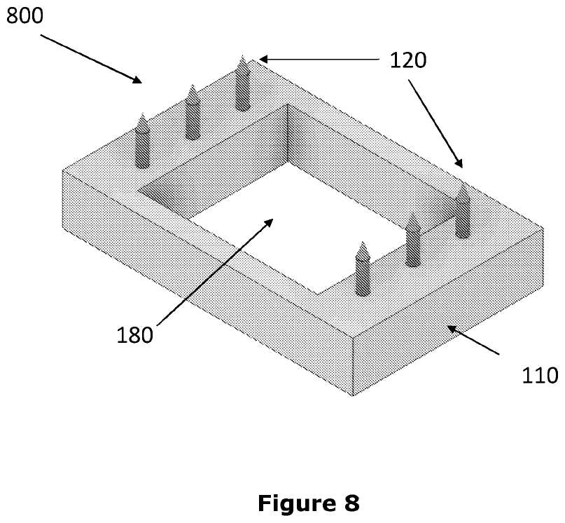

### Example Method—Producing Tissue Engineered Murine Muscle Using an Insert and Anchors

The murine immortalised myoblast cell line C2C12 (ATCC) was used to demonstrate that tissue engineered muscle can be grown on the insert.

### Cell Expansion:

Growth medium (gm) was prepared as follows: A medium (e.g. DMEM) supplemented with 20% foetal bovine serum and 1% Penicillin-Streptomycin.

Differentiation medium (dm) was prepared as follows: DMEM+1% pen-strep with 2% horse serum.

Cells were seeded at a density of ˜5000 cells/cm² in gm and incubated until they reached≤70% confluence.

The gm was renewed the next day if plated from a cryopreservation tube, otherwise it was changed every 2 days.

### Subculturing:

Once the cells were ≤70% confluent, gm was removed, the cells were washed with DPBS (—Ca/—Mg) and then cells were detached using 0.25% (w/v) Trypsin-0.53 mM EDTA solution.

Cells were incubated with trypsin at 37° C. for 5 min and checked under the microscope to confirm that the cells were floating. An equivalent volume of complete pre-warmed gm was added to neutralise the trypsin (e.g., 3 ml of gm if used 3 ml of trypsin). The cell suspension was mixed thoroughly e.g., by pipetting up and down 10× in a gently manner to avoid the formation of foam.

### Cell Counting:

100 μl of cells were transferred into an Eppendorf tube and 400 μl Trypan blue was added to stain dead cells. After incubating at room temperature for a few minutes 10 μl was removed and loaded onto an hemacytometer (e.g. Neubauer cell counting chamber). Cell concentration was then calculated.

### Preparation of Hydrogel

All plasticware was pre-frozen and work was conducted on ice when preparing the hydrogel.

Approximately 3.0 mg/ml collagen I concentration was used as follows:

| Initial Col I concentration | Volume col I | Volume 10x PBS |
|---|---|---|
| 3.78 mg/ml | 0.397 ml | 0.103 ml |
| 3.48 mg/ml | 0.431 ml | 0.069 ml |
| [ | | |

Matrigel was used undiluted if protein concentration is at 8 mg/ml. 65% v/v type I Rat tail collagen was mixed with 10% v/v MEM, the solution was then neutralised by dropwise addition of 1M NaOH until colour turns pink. pH strips were used to check that the pH was ˜7. 20% v/v of Matrigel was added. The hydrogel was kept in ice for max 2 h before using.

### Preparation of Cell/Hydrogel Mixture:

The cell suspension was centrifuged for 5 minutes at 1000 rpm. The supernatant was discarded, and the pellet resuspended in a solution of Matrigel and collagen.

The cell suspension was mixed thoroughly e.g., by pipetting up and down 10× in a gently manner to avoid the formation of foam. The cell hydrogel mixture was kept in ice until seeding.

Prepare Inserts with Anchor:

The anchors were pierced (2 holes in each) using a syringe needle and one was fitted in each of the first pair of pillars of the insert. The insert and anchors were sterilised. The insert was placed in a well of a well-plate under aseptic conditions.

Load Insert with Hydrogel and Cells:

The cell-hydrogel mixture was thoroughly mixed prior to pipetting 200 μl of the solution into the central groove of the insert. First a small drop of gel was pipetted under each anchor. Then the anchors were pressed down and released several times. Finally, the top of the anchor and groove is filled with hydrogel, by moving the pipette back and forth to create uniform strip of cells.

The baseplate with culture plates were placed in a 37° C. incubator for 2 h to allow the hydrogel to polymerise.

After the gel has set, 3 ml of gm was carefully added to each well.

Once myoblasts reached ≥90% confluence (depending on the cell line), the cells were washed with PBS and the media changed to dm.

Another hole of the anchor can be used to stretch the tissue engineered muscle and enhance functional maturation if needed. Alternatively or additionally, the tissue engineered muscle can be moved to pillars 3 and 4 for culture in suspension.

Myotube formation occurred over 12 days.

Differentiation medium was changed every other day.

1. An insert for culturing tissue comprising:
a base comprising a groove configured to provide a guide for adding cells to be cultured;
a first pillar upstanding from the base and configured to support a first anchor; and
a second pillar upstanding from the base and configured to support a second anchor;
wherein the insert is configured so that cells may be cultured to form tissue between the first and second pillars using the anchors for support.

2. The insert of claim 1, wherein the groove extends from a bottom of the first pillar to a bottom of the second pillar.

3-5. (canceled)

6. The insert of claim 1, wherein the first and second pillars comprise a biodegradable material.

7. (canceled)

8. The insert of claim 1, wherein the position of at least one of the first pillar or the second pillar is adjustable.

9. (canceled)

10. The insert of claim 8, wherein the positions of the anchors are configured to be changed manually, automatically, or semi-automatically.

11. (canceled)

12. (canceled)

13. (canceled)

14. (canceled)

15. (canceled)

16. The insert according to claim 8, further comprising:
a third and a fourth pillar configured to support anchors, and wherein the pillars are spaced equidistant around the base of the insert.

17. A system for culturing tissue, comprising:
an insert for culturing tissue comprising:
a base comprising a groove configured to provide a guide for adding cells to be cultured;
a first pillar upstanding from the base and configured to support a first anchor; and
a second pillar upstanding from the base and configured to support a second anchor; and
a first anchor and a second anchor; and
a plate comprising at least one well,
wherein the insert is configured so that cells may be cultured to form tissue between the first and second pillars using the anchors for support, and
wherein the at least one well contains the insert, and
wherein a hydrogel is suspended in the insert.

18. The system of claim 17, wherein the first anchor and the second anchor comprise a biocompatible non-woven textile.

19. The system of claim 17, wherein the first anchor and the second anchor are configured to receive the first and second pillars.

20. (canceled)

21. (canceled)

22. (canceled)

23. The system of claim 17, wherein the insert further comprises:
a third pillar upstanding from the base and configured to support an anchor; and
a fourth pillar upstanding from the base and configured to support an anchor.

24. (canceled)

25. (canceled)

26. (canceled)

27. The system of claim 17 further comprising:
a secondary insert, wherein the secondary insert comprises:
a body comprising an aperture;
a first pair of pillars upstanding from the body and configured to support a first pair of anchors; and
a second pair of pillars upstanding from the body and configured to support a second pair of anchors;
wherein the secondary insert is configured to receive one or more pairs of anchors removed from the pillars of the primary insert during growth of the tissue.

28. The system of claim 27, wherein the first and second pillars of the secondary insert comprise a biodegradable material.

29. The system of claim 27, wherein the third and fourth pillar comprise a spacer at the base of each pillar, the spacer configured to support the anchor above the base of the insert.

30. The system of claim 23, wherein the third and fourth pillars are disposed along a plane perpendicular to the groove.

31. The system of claim 23, wherein the insert further comprises a force transducer.

32. The system of claim 30, wherein the force transducer is one of a physical sensor, an optical sensor, a RF sensor, an acoustic sensor, or a Fibre optic sensor.

33. The system of claim 23, wherein the groove further comprises a recess.

34. The system of claim 23, wherein the first and second pillars are made of a biocompatible material, optionally wherein the biocompatible material is a biocompatible resin.

35. An in vitro method of culturing tissue, the method comprising:
seeding cells in a groove formed in a base of an insert, the groove extending between a first pillar and a second pillar, each pillar configured to support an anchor; and
securing an anchor to the first and the second pillars,
wherein the insert is configured so that cells may be cultured to form tissue between the first and second pillars using the anchors for support.

36. The method of claim 35, further comprising:
suspending a hydrogel in a well of a plate, the plate configured to receive the insert.
